(19) United States
(12) Patent Application Publication
(10) Pub. No.: US 2024/0279189 A1
(43) Pub. Date: Aug. 22, 2024

(54) METHOD FOR PRODUCING BENZOXAZINE COMPOUND

(71) Applicant: HONSHU CHEMICAL INDUSTRY CO., LTD., Chuo-ku, Tokyo (JP)

(72) Inventor: Yoshimi UTAKA, Wakayama-shi (JP)

(21) Appl. No.: 18/262,151

(22) PCT Filed: Jan. 24, 2022
(86) PCT No.: PCT/JP2022/002310

§ 371 Date: Jul. 19, 2023

(30) Foreign Application Priority Data
Jan. 29, 2021 (JP) 2021-013396
Jan. 29, 2021 (JP) 2021-013397
Jan. 29, 2021 (JP) 2021-013398

(51) Int. Cl. C07D 265/16

(52) U.S. Cl. CPC C07D 265/16

(57) ABSTRACT

A method for producing a benzoxazine compound represented by general formula (3), includes performing a reaction of a bisphenol compound represented by general formula (1), a formaldehyde, and an amine compound represented by general formula (2) in a specific temperature range:

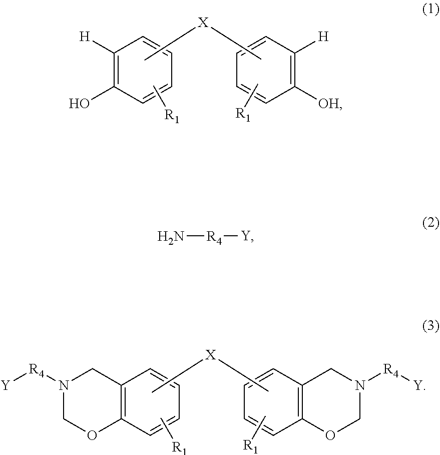

### TECHNICAL FIELD

The present invention relates to a method for producing a benzoxazine compound. The invention particularly relates to a method for producing a benzoxazine compound having benzoxazine rings at both ends of a linking group and further having a hydroxy group or a thiol group.

### BACKGROUND ART

Benzoxazine compounds, which are compounds synthesized by reacting a phenol, an amine, and formaldehyde, are known as thermosetting resin raw materials that, when heated, undergo ring-opening polymerization of a benzoxazine ring to cure without producing any volatile by-products, and are used as raw materials of a molded body usable as a material for an insulating substrate, a liquid crystal alignment agent, a resin composition for semiconductor sealing, and the like. In these applications, heat resistance excellent in stability and reliability at high temperatures is required.

On the other hand, benzoxazine compounds typically have relatively high curing temperatures, and to achieve lower polymerization temperatures, catalysts, polymerization accelerators, and in addition highly reactive benzoxazine compounds have recently been developed. Among the highly reactive benzoxazine compounds, a benzoxazine composition that can be cured in an environmentally friendly process at relatively low temperatures in short time periods and contains a hydroxy group or a nitrogen-containing heterocycle has been reported (PTL 1).

Examples of known methods for synthesizing a benzoxazine compound containing a hydroxy group include a synthesis method in which bisphenol A, an alcohol amine monomer, and paraformaldehyde, which are raw materials, are mixed at one time together with a solvent and allowed to react (PTL 2) and a synthesis method in which a mixed solution of paraformaldehyde and ethanolamine is first prepared, and then a solution of bisphenol A is added and allowed to react (NPL 1). It has been reported that in these methods, the synthesis is performed at a high temperature of 90° C. or higher, and the product yield is relatively high.

### CITATION LIST

### Patent Literature

PTL 1: Japanese Unexamined Patent Application Publication (Translation of PCT Application) No. 2011-530570
PTL 2: Chinese Patent Application Publication No.

### Non Patent Literature

NPL 1: E. Gilbert et al., J. Polym. Res. 2018, Vol. 25, p. 114.

### SUMMARY OF INVENTION

### Technical Problem

The present inventors have attempted to synthesize the benzoxazine compound having a hydroxy group or a thiol group according to the present invention with reference to the above production methods known in the art, and found that a reaction solution during the reaction is solidified, so that the reaction cannot be completed, as in Comparative Examples described later and that problems occur such as decrease in reaction selectivity and solidification of a solution containing a target compound during the operation of collecting the target compound, so that the benzoxazine compound cannot be produced or cannot be efficiently produced.

It is an object of the present invention to provide a method for producing a target benzoxazine compound having a hydroxy group or a thiol group in high purity and with efficiency.

### Solution to Problem

To achieve the above object, the present inventors have conducted intensive studies and found that contrary to the descriptions of the above related art literatures, the target benzoxazine compound can be synthesized with high selectivity by reacting a bisphenol compound, a formaldehyde, and an amine in a lower temperature range, thereby completing the present invention.

The present invention is as follows.
1. A method for producing a benzoxazine compound represented by general formula (3), the method including performing a reaction of a bisphenol compound represented by general formula (1), a formaldehyde, and an amine compound represented by general formula (2) at 10° C. or higher and 80° C. or lower.

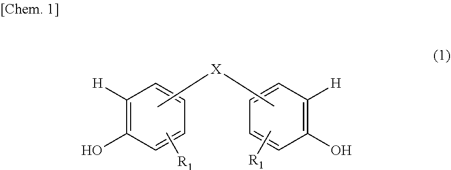

(In the formula, R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b).)

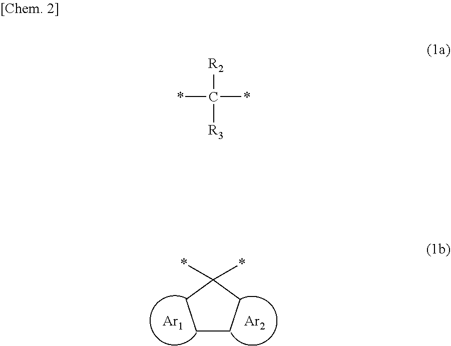

(In general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R₃ are optionally bonded to each other to together form a cycloalkylidene group having 5 to 20 carbon atoms, Ar₁ and Ar₂ each independently represent an aryl group having 6 to 12 carbon atoms, and * represents a bonding position.)

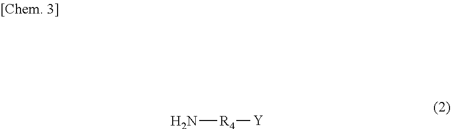

(In the formula, R₄ represents a divalent group having 1 to 10 carbon atoms, and Y represents a hydroxy group or a thiol group.)

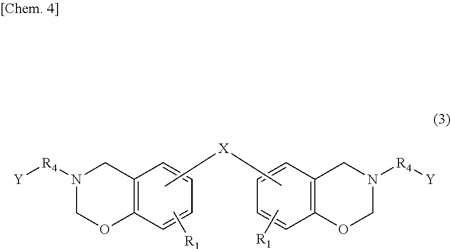

(In the formula, R₁ and X are as defined in general formula (1), and R₄ and Y are as defined in general formula (2).)

### Advantageous Effects of Invention

According to the method for producing a benzoxazine compound according to the present invention, a benzoxazine compound having a hydroxy group or a thiol group can be produced with high selectivity. The benzoxazine compound obtained by the method contains the benzoxazine compound serving as an active component in a large amount and thus is very useful as a raw material of a curable resin.

Furthermore, the method for producing a benzoxazine compound according to the present invention is very useful because viscosity increase or solidification of a solution containing the target compound after completion of the reaction can be suppressed and thus the benzoxazine compound can be efficiently produced.

### DESCRIPTION OF EMBODIMENTS

### <Method for Producing Benzoxazine Compound According to Present Invention>

A method for producing a benzoxazine compound according to the present invention includes performing a reaction of a bisphenol compound represented by general formula (1), a formaldehyde, and an amine compound represented by general formula (2) in a specific temperature range, and a benzoxazine compound represented by general formula (3) is the target compound of the production method.

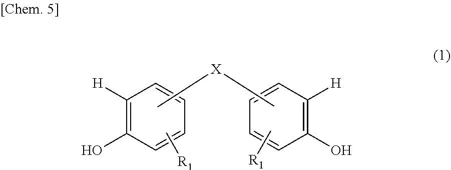

(In the formula, R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b).)

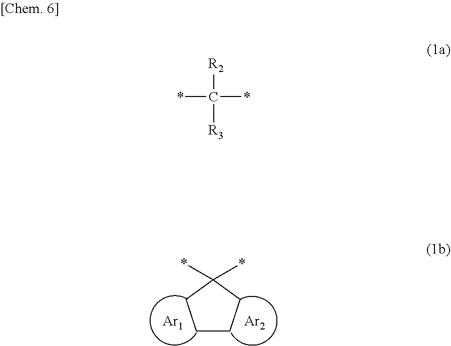

(In general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R₃ are optionally bonded to each other to together form a cycloalkylidene group having 5 to 20 carbon atoms, Ar₁ and Ar₂ each independently represent an aryl group having 6 to 12 carbon atoms, and * represents a bonding position.)

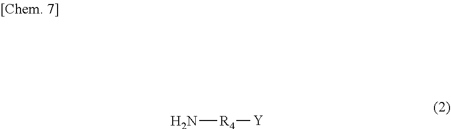

(In the formula, R₄ represents a divalent group having 1 to 10 carbon atoms, and Y represents a hydroxy group or a thiol group.)

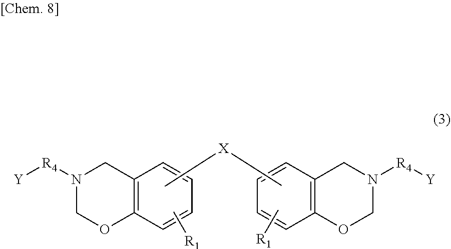

(In the formula, R₁ and X are as defined in general formula (1), and R₄ and Y are as defined in general formula (2).)

R₁ in general formulae (1) and (3) is preferably a hydrogen atom or an alkyl group having 1 to 4 carbon atoms, more preferably a hydrogen atom or an alkyl group having 1 carbon atom (methyl group), particularly preferably a hydrogen atom. When R₁ is not a hydrogen atom, the bonding position thereof is preferably the ortho position to each hydroxy group in general formula (1), and is preferably the ortho position on the benzene ring relative to the oxygen atom of each benzoxazine ring in general formula (3).

When X in general formulae (1) and (3) is represented by general formula (1a), R₂ and R₃, independently of each other, are more preferably hydrogen, an alkyl group having 1 to 6 carbon atoms, an alkyl halide group having 1 to 6 carbon atoms, or an aryl group having 6 to 12 carbon atoms, still more preferably hydrogen, an alkyl group having 1 to 4 carbon atoms, a trifluoromethyl group, or an aryl group having 6 to 8 carbon atoms, particularly preferably hydrogen, an alkyl group having 1 to 4 carbon atoms, or a phenyl group.

R₂ and R₃ may be bonded to each other to together form a cycloalkylidene group having 5 to 20 carbon atoms. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms.

Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), a cycloheptylidene group (7 carbon atoms), a bicyclo[2.2.1]heptane-2,2-diyl group (7 carbon atoms), a 1,7,7-trimethylbicyclo[2.2.1]heptane-2,2-diyl group (10 carbon atoms), a 4,7,7-trimethylbicyclo[2.2.1]heptane-2,2-diyl group (10 carbon atoms), a tricyclo[5.2.1.0^{2,6}]decane-8,8-diyl group (10 carbon atoms), a 2,2-adamantylidene group (10 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), more preferred are a cyclohexylidene group (6 carbon atoms), a 3,3,5-trimethylcyclohexylidene group (9 carbon atoms), and a cyclododecanylidene group (12 carbon atoms), and particularly preferred are a cyclohexylidene group (6 carbon atoms) and a 3,3,5-trimethylcyclohexylidene group (9 carbon atoms).

When X in general formulae (1) and (3) is represented by general formula (1b), Ar₁ and Ar₂ are preferably each independently a benzene ring or a naphthalene ring, and Ar₁ and Ar₂ are more preferably each a benzene ring. For example, when Ar₁ and Ar₂ are each a benzene ring, the group represented by general formula (1b) is a fluorenylidene group.

The position of bonding of X in general formula (3) to the two benzoxazine rings is preferably the ortho or para position on the benzene ring relative to the oxygen atom of each benzoxazine ring, and the position of bonding of X on the benzene ring in general formula (1), which is the material of general formula (3), is also preferably the ortho or para position relative to each hydroxy group.

R₄ in general formulae (2) and (3) is a divalent group having 1 to 10 carbon atoms, and specific examples include linear or branched alkylene groups having 1 to 10 carbon atoms or alkylene groups including a cyclic alkane, such as a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group; alkylidene groups having 1 to 10 carbon atoms such as an ethylidene group, a propylidene group, an isopropylidene group, a butylidene group, a cyclopentylidene group, and a cyclohexylidene group; a phenylene group; and divalent groups containing a benzene ring and having 1 to 10 carbon atoms, such as groups represented by the following formulae.

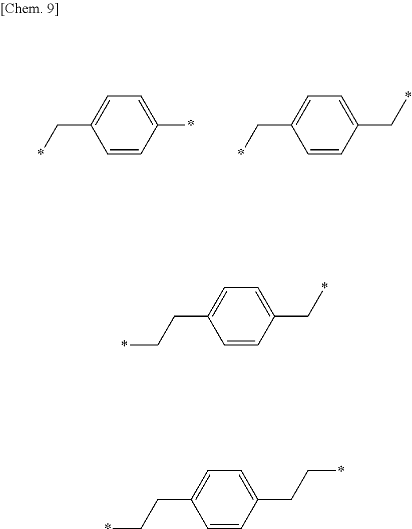 [SMILES: *Cc1ccc(*)cc1 ; *Cc1ccc(C*)cc1 ; *CCc1ccc(C*)cc1 ; *CCc1ccc(CC*)cc1]

(In the formulae, * represents a bonding position.)

Of these, R₄ is preferably a linear or branched alkylene group having 1 to 10 carbon atoms, an alkylene group including a cyclic alkane, or an alkylidene group having 1 to 10 carbon atoms, more preferably a linear or branched alkylene group having 1 to 10 carbon atoms or an alkylene group including a cyclic alkane, still more preferably a linear or branched alkylene group having 1 to 6 carbon atoms or an alkylene group including a cyclic alkane, particularly preferably a linear or branched alkylene group having 1 to 4 carbon atoms.

Specific examples of the benzoxazine compound represented by general formula (3), which is the target compound of the production method according to the present invention, include compounds (p-1) to (p-6) having the following chemical structures.

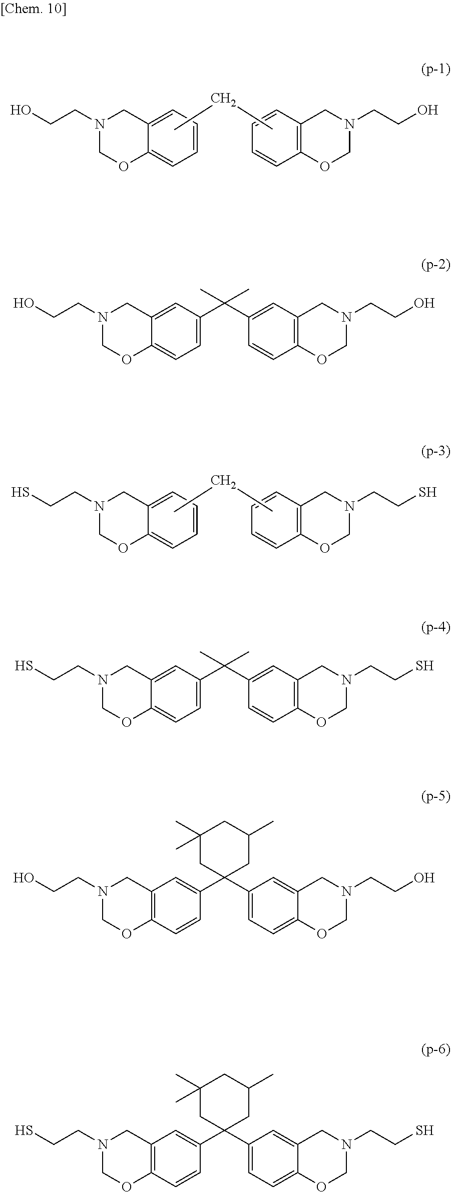

### <Starting Material: Bisphenol Compound Represented by General Formula (1)>

Specific examples of the bisphenol compound represented by general formula (1), which is a starting material in the method for producing a benzoxazine compound according to the present invention, include bisphenol F (bis(2-hydroxyphenyl)methane, 2-hydroxyphenyl-4-hydroxyphenylmethane, bis(4-hydroxyphenyl)methane), bisphenol E (1,1-bis(4-hydroxyphenyl)ethane), bisphenol A (2,2-bis(4-hydroxyphenyl)propane), bisphenol C (2,2-bis(4-hydroxy-3-methylphenyl)propane), 2,2-bis(4-hydroxyphenyl)-4-methylpentane, 4,4′-dihydroxybiphenyl, 4,4′-dihydroxy-3,3′-dimethylbiphenyl, bis(4-hydroxyphenyl) ether, 4,4′-dihydroxybenzophenone, bis(4-hydroxyphenyl)sulfone, bis(4-hydroxyphenyl) sulfide, 1,1-bis(4-hydroxyphenyl)-1-phenylethane, 1,1-bis(4-hydroxyphenyl)-1-naphthylethane, 2,2-bis(4-hydroxyphenyl)hexafluoropropane, bisphenol M (1,3-bis(2-(4-hydroxyphenyl)-2-propyl)benzene), bisphenol Z (1,1-bis(4-hydroxyphenyl)cyclohexane), bisphenol TMC (1,1-bis(4-hydroxyphenyl)-3,3,5-trimethylcyclohexane), 1,1-bis(4-hydroxyphenyl)cyclododecane, 2,2-bis(4-hydroxyphenyl)adamantane, and 9,9-bis(4-hydroxy-3-methylphenyl) fluorene.

### <Starting Material: Formaldehyde>

Specific examples of the formaldehyde, which is a starting material in the novel method for producing a benzoxazine compound according to the present invention, include an aqueous formaldehyde solution, 1,3,5-trioxane, and paraformaldehyde.

### <Starting Material: Amine Compound Represented by General Formula (2)>

Specific examples of the amine compound represented by general formula (2), which is a starting material in the method for producing a benzoxazine compound according to the present invention, include the following compounds.

Specific examples of cases where “Y” in general formula (2) is a hydroxy group include methanolamine, 2-aminoethanol, 1-amino-2-propanol, 2-amino-1-methylethanol, 2-amino-2-methylethanol, 3-amino-1-propanol, 4-amino-1-butanol, 2-amino-1-butanol, 4-amino-2-butanol, 5-amino-1-pentanol, 6-amino-1-hexanol, 7-amino-1-heptanol, valinol, 2-aminophenol, 3-aminophenol, 4-aminophenol, and 4-aminobenzyl alcohol. Of these, 2-aminoethanol, 2-amino-1-methylethanol, 2-amino-2-methylethanol, 3-amino-1-propanol, 2-aminophenol, 3-aminophenol, and 4-aminophenol are preferred, 2-aminoethanol, 2-aminophenol, 3-aminophenol, and 4-aminophenol are more preferred, and 2-aminoethanol is particularly preferred.

Specific examples of cases where “Y” in general formula (2) is a thiol group include 2-aminoethanethiol, 3-amino-1-propanethiol, 2-amino-1-methylethanethiol, 2-amino-2-methylethanethiol, 5-amino-1-pentanethiol, 6-amino-1-hexanethiol, 2-aminothiophenol, 3-aminothiophenol, 4-aminothiophenol, and 4-aminobenzyl mercaptan. Of these, 2-aminoethanethiol, 3-amino-1-propanethiol, 2-aminothiophenol, 3-aminothiophenol, and 4-aminothiophenol are preferred, 2-aminoethanethiol, 2-aminothiophenol, 3-aminothiophenol, and 4-aminothiophenol are more preferred, and 2-aminoethanethiol is particularly preferred.

### <Amount of Starting Material Used>

In the production method according to the present invention, the amount of the formaldehyde used is preferably in the range of 4.0 to 20.0 mol, more preferably in the range of 4.0 to 16.0 mol, still more preferably in the range of 4.0 to 12.0 mol, relative to 1 mol of the bisphenol compound represented by general formula (1).

In the production method according to the present invention, the amount of the amine compound represented by general formula (2) used is preferably in the range of 2.0 to 10.0 mol, more preferably in the range of 2.0 to 8.0 mol, still more preferably in the range of 2.0 to 6.0 mol, relative to 1 mol of the bisphenol compound represented by general formula (1).

### <Catalyst>

In the production method according to the present invention, a catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

### <Solvent>

In the production method according to the present invention, the reaction is typically performed in the presence of a solvent. The solvent is not particularly limited as long as it does not inhibit the reaction, and preferred examples include toluene, xylene, ethyl acetate, butyl acetate, chloroform, dichloromethane, tetrahydrofuran, and dioxane. These solvents can be used alone or in combination. The amount of solvent used is not particularly limited as long as the reaction is not hindered, and is typically in the range of 0.5 to 5 times, preferably in the range of 1 to 3 times the amount of the bisphenol compound represented by general formula (1) on a weight basis.

### <Reaction Temperature>

In the production method according to the present invention, the reaction is performed at 10° C. or higher and 80° C. or lower. The reaction temperature is preferably 20° C. or higher and 75° C. or lower, more preferably 20° C. or higher and 70° C. or lower, still more preferably 20° C. or higher and 60° C. or lower, particularly preferably 20° C. or higher and 40° C. or lower.

Performing the reaction in this temperature range is very useful because improvement in reaction selectivity of the benzoxazine compound represented by general formula (3), which is the target compound, suppression of formation of by-product high-molecular-weight components, and suppression of viscosity increase or solidification of a solution after the reaction can be achieved and thus the target benzoxazine compound can be efficiently produced in high purity.

### <Reaction Pressure>

In the production method according to the present invention, the reaction may be performed under normal pressure conditions, or may be performed under increased pressure or reduced pressure.

In another embodiment, a process of removing water derived from the raw materials or water generated during the reaction out of the system may be included. The process of removing water generated from a reaction solution is not particularly limited and can be performed by distilling the generated water azeotropically with the solvent system in the reaction solution. The generated water can be removed out of the reaction system by using, for example, an isobaric dropping funnel equipped with a cock, a Dimroth condenser, or a Dean-Stark apparatus.

In the production method according to the present invention, the method of mixing the bisphenol compound represented by general formula (1), the formaldehyde, and the amine compound represented by general formula (2) used as raw materials is not particularly limited. Examples include (i) a method in which a mixture containing the bisphenol compound represented by general formula (1) and the formaldehyde is mixed with the amine compound represented by general formula (2) to perform the reaction and (ii) a method in which a mixture containing the formaldehyde and the amine compound represented by general formula (2) is mixed with the bisphenol compound represented by general formula (1). These mixtures may contain the solvent and the catalyst described above. The method of mixing the catalyst is not particularly limited, but the catalyst is preferably mixed before the amine compound represented by general formula (2) is mixed.

In the production method according to the present invention, the method of mixing a mixture of raw materials with the rest of the raw materials is not particularly limited, but from the viewpoint of reaction selectivity and suppression of formation of by-product high-molecular-weight components, continuous or intermittent mixing is preferred to mixing all at once.

### <After Completion of Reaction>

From the final reaction mixture obtained by the production method according to the present invention, the benzoxazine compound represented by general formula (3) can be collected by a known method. For example, the remaining raw materials and solvent may be distilled off from the final reaction mixture to thereby obtain the target as a residual liquid. Other possible methods include adding the residual liquid to a poor solvent to obtain the target as a precipitate, and adding a solvent to the reaction mixture to cause crystallization and performing filtration to obtain the target as powder or particles. The benzoxazine compound collected by any of these methods can be made into a high-purity product by, for example, standard purification means such as washing with a solvent or water or recrystallization.

### EXAMPLES

The present invention will now be described more specifically with reference to Examples.

### <Analysis Method>

### 1. Reaction Solution Composition and Purity Analysis (Gel Permeation Chromatography: GPC)

The purity of benzoxazine compounds synthesized by the production method according to the present invention was defined as the area percentage of the benzoxazine compounds determined by this analysis.
Apparatus: HLC-8320/manufactured by Tosoh Corporation
Detector: differential refractometer (RI)

### [Measurement Conditions]

Flow rate: 1 mL/min
Eluate: tetrahydrofuran
Temperature: 40° C.
Wavelength: 254 nm
Measurement sample: One gram of a benzoxazine compound-containing composition was 200-fold diluted with tetrahydrofuran.

### <Example 1> (Synthesis of Compound p-1)

In a 1 L four-necked flask equipped with a thermometer, a stirrer, a condenser, and a dropping funnel, 97 g (0.48 mol) of bisphenol F (binuclear structure content, 90.1 wt %; isomer ratio thereof: bis(2-hydroxyphenyl)methane, 18.8 wt %; 2-hydroxyphenyl-4-hydroxyphenylmethane, 49.3 wt %; and bis(4-hydroxyphenyl)methane, 31.9 wt %; polynuclear structure content, 9.9 wt %), 62 g of 94% paraformaldehyde, and 121 g of toluene were loaded. After the reaction vessel was purged with nitrogen, the temperature of the mixed solution was adjusted to 70° C. While maintaining the temperature, 60 g of 2-aminoethanol was added dropwise into the four-necked flask using a dropping funnel over 2 hours. After completion of the dropwise addition, stirring was further performed at 70° C. for 3 hours. The composition of the reaction solution was analyzed by GPC according to the above analysis method, revealing that the percentage of the target compound present in the reaction solution was 51 area %.

After completion of the reaction, toluene and water were removed by reduced-pressure distillation at 70° C. The pressure during the distillation was gradually reduced so as to finally reach 4.8 kPa. The composition containing the target compound was taken out, solidified by cooling, then pulverized, and dried at 60° C. and 1.5 kPa to obtain 173 g of the target compound (purity: 53%, compounds with molecular weights higher than that of the target compound: 47 area %).

The results of 1H-NMR analysis confirmed that a target benzoxazine compound (p-1) was obtained.

¹H-NMR analysis (400 MHZ, solvent: CDCl₃, reference material: tetramethylsilane)

2.43-2.72 (2H, brm), 2.71-3.16 (4H, m), 3.41-4.09 (12H, m), 4.69-5.01 (4H, m), 6.49-7.07 (6H, m).

### <Example 2> (Synthesis of Compound p-1)

In a 1 L four-necked flask equipped with a thermometer, a stirrer, a condenser, and a dropping funnel, 221.5 g (1.11 mol) of bisphenol F (the same product as in Example 1), 173.5 g of 94% paraformaldehyde, and 409.8 g of toluene were loaded. After the reaction vessel was purged with nitrogen, the temperature of the mixed solution was adjusted to 30° C. While maintaining the temperature, 135.2 g of 2-aminoethanol was added dropwise into the four-necked flask using a dropping funnel over 2 hours. After completion of the dropwise addition, stirring was further performed at 30° C. for 1 hour. The composition of the reaction solution was analyzed by GPC according to the above analysis method, revealing that the percentage of the target compound present in the reaction solution was 77 area %.

### <Comparative Example 1> (Synthesis of Compound p-1)

In a 1 L four-necked flask equipped with a thermometer, a stirrer, a condenser, and a dropping funnel, 97 g (0.48 mol) of bisphenol F (the same product as in Example 1), 62 g of 94% paraformaldehyde, and 121 g of toluene were loaded. After the reaction vessel was purged with nitrogen, the temperature of the mixed solution was adjusted to 90° C. While maintaining the temperature, 60 g of 2-aminoethanol was added dropwise into the four-necked flask using a dropping funnel over 2 hours. After completion of the dropwise addition, stirring was further performed at 90° C. for 3 hours. The composition of the reaction solution was analyzed by GPC according to the above analysis method, revealing that the percentage of the target compound present in the reaction solution was 47 area %.

After completion of the reaction, an attempt was made to remove toluene and water by reduced-pressure distillation at 70° C., but the solution in the reaction vessel solidified, so that stirring could not be continued. Therefore, the composition containing the target compound could not be taken out from the reaction vessel.

### <Example 3> (Synthesis of Compound p-2)

In a 1 L four-necked flask equipped with a thermometer, a stirrer, a condenser, and a dropping funnel, 100 g (0.44 mol) of bisphenol A, 56 g of 94% paraformaldehyde, and 184 g of toluene were loaded. After the reaction vessel was purged with nitrogen, the temperature of the mixed solution was adjusted to 70° C. While maintaining the temperature, 53 g of 2-aminoethanol was added dropwise into the four-necked flask using a dropping funnel over 2 hours. After completion of the dropwise addition, stirring was further performed at 70° C. for 9.5 hours. The composition of the reaction solution was analyzed by GPC according to the above analysis method, revealing that the percentage of the target compound present in the reaction solution was 52 area %.

After completion of the reaction, toluene and water were removed by reduced-pressure distillation at 70° C. The pressure during the distillation was gradually reduced so as to finally reach 20 kPa. The composition containing the target compound was taken out to obtain 187 g of the target compound (purity: 54%, compounds with molecular weights higher than that of the target compound: 46 area %).

The results of ¹H-NMR analysis confirmed that a target benzoxazine compound (p-2) was obtained.

¹H-NMR analysis (400 MHZ, solvent: CDCl₃, reference material: tetramethylsilane)

1.14-1.96 (6H, m), 2.45-2.77 (2H, brm), 2.78-3.18 (4H, m), 3.28-4.19 (10H, m), 4.70-5.14 (4H, m), 6.56-7.13 (6H, m).

### <Example 4> (Synthesis of Compound p-3)

In a 500 mL four-necked flask equipped with a thermometer, a stirrer, a condenser, and a dropping funnel, 31 g (0.15 mol) of bisphenol F (the same product as in Example 1), 20 g of 94% paraformaldehyde, and 57 g of toluene were loaded. After the reaction vessel was purged with nitrogen, the temperature of the mixed solution was adjusted to 60° C. While maintaining the temperature, 24 g of 2-aminoethanethiol was added dropwise into the four-necked flask using a dropping funnel over 1 hour. After completion of the dropwise addition, stirring was further performed at 60° C. for 2 hours. The composition of the reaction solution was analyzed by GPC according to the above analysis method, revealing that the percentage of the target compound present in the reaction solution was 41 area %.

After completion of the reaction, toluene and water were removed by reduced-pressure distillation at 50° C. The pressure during the distillation was gradually reduced so as to finally reach 2.4 kPa. The composition containing the target compound was taken out to obtain 59 g of the target compound (purity: 41%, compounds with molecular weights higher than that of the target compound: 59 area %).

The results of 1H-NMR analysis confirmed that a target benzoxazine compound (p-3) was obtained.

¹H-NMR (400 MHZ, solvent: CDCl₃, reference material: tetramethylsilane)

1.32-1.95 (2H, brm), 2.91-3.05 (4H, m), 3.07-3.22 (4H, m), 3.64-4.13 (10H, m), 6.66-7.12 (6H, m).

### <Example 5> (Synthesis of Compound p-3)

The reaction was performed in the same manner as in Example 4 except that a 1 L four-necked flask equipped with a thermometer, a stirrer, and a condenser, 97 g (0.48 mol) of bisphenol F (the same product as in Example 1), 62 g of 94% paraformaldehyde, 75 g of 2-aminoethanethiol, and 180 g of toluene were used, the temperature before the dropwise addition of amine was adjusted to 50° C., and stirring was further performed at 50° C. for 1 hour after completion of the dropwise addition of amine. The composition of the reaction solution was analyzed by GPC according to the above analysis method, revealing that the percentage of the target compound present in the reaction solution was 65 area %.

After completion of the reaction, toluene and water were removed by reduced-pressure distillation at 50° C. The pressure during the distillation was gradually reduced so as to finally reach 1.5 kPa. The composition containing the target compound was taken out, solidified by cooling, then pulverized, and vacuum-dried at 60° C. and 1.5 kPa to obtain 208 g of the target compound (purity: 56%, compounds with molecular weights higher than that of the target compound: 44 area %).

### <Example 6> (Synthesis of Compound p-3)

The reaction was performed in the same manner as in Example 4 except that a 1 L four-necked flask equipped with a thermometer, a stirrer, and a condenser, 97 g (0.48 mol) of bisphenol F (the same product as in Example 1), 74 g of 94% paraformaldehyde, 75 g of 2-aminoethanethiol, and 180 g of toluene were used, the temperature before the dropwise addition of amine was adjusted to 30° C., and stirring was further performed at 30° C. for 3 hours after completion of the dropwise addition of amine. The composition of the reaction solution was analyzed by GPC according to the above analysis method, revealing that the percentage of the target compound present in the reaction solution was 88 area %.

After completion of the reaction, alkali washing was performed using a 3% aqueous sodium hydroxide solution, and water washing was then performed until the pH of the reaction solution became 7 or less. Thereafter, toluene and water were removed by reduced-pressure distillation at 30° C. The pressure during the distillation was gradually reduced so as to finally reach 2.3 kPa. After the solvent was removed to some extent, the remaining solvent was further removed at 90° C. and 2.8 kPa. The composition containing the target compound was taken out, solidified by cooling, and then pulverized to obtain 156 g of the target compound (purity: 75%, compounds with molecular weights higher than that of the target compound: 25 area %).

### <Example 7> (Synthesis of Compound p-3)

In a 500 mL four-necked flask equipped with a thermometer, a stirrer, and a condenser, 30 g of 94% paraformaldehyde, 36 g of 2-aminoethanethiol, and 88 g of toluene were loaded. Then, after the reaction vessel was purged with nitrogen, the mixed solution was heated to 60° C. and mixed with 47 g (0.23 mol) of bisphenol F (the same product as in Example 1) while maintaining the temperature. After completion of the mixing, stirring was further performed at 60° C. for 3 hours. The composition of the reaction solution was analyzed by GPC according to the above analysis method, revealing that the percentage of the target compound present in the reaction solution was 69 area %.

### <Comparative Example 2> (Synthesis of Compound p-3)

The reaction was performed in the same manner as in Example 4 except that a 1 L four-necked flask equipped with a thermometer, a stirrer, and a condenser, 97 g (0.48 mol) of bisphenol F (the same product as in Example 1), 62 g of 94% paraformaldehyde, 75 g of 2-aminoethanethiol, and 180 g of toluene were used, and the temperature before the dropwise addition of amine was adjusted to 90° C. The reaction solution solidified during the dropwise addition of amine, so that stirring could not be continued, and the reaction could not be completed.

### <Example 8> (Synthesis of Compound p-5)

In a 1 L four-necked flask equipped with a thermometer, a stirrer, a condenser, and a dropping funnel, 124 g (0.4 mol) of 1,1-bis(4-hydroxyphenyl)-3,3,5-trimethylcyclohexane, 63 g of 92% paraformaldehyde, and 230 g of toluene were loaded. After the reaction vessel was purged with nitrogen, the temperature of the mixed solution was adjusted to 30° C. While maintaining the temperature, 49 g of 2-aminoethanol was added dropwise into the four-necked flask using a dropping funnel over 2 hours. After completion of the dropwise addition, stirring was further performed at 30° C. for 4 hours. The composition of the reaction solution was analyzed by GPC according to the above analysis method, revealing that the percentage of the target compound present in the reaction solution was 79 area %.

After completion of the reaction, alkali washing was performed using a 3% aqueous sodium hydroxide solution, after which 350 g of toluene was added, and water washing was performed until the pH of the water-washed solution became 7 or less. Thereafter, toluene and water were removed by reduced-pressure distillation at 60° C. The pressure during the distillation was gradually reduced so as to finally reach 4.8 kPa. After the solvent was removed to some extent, the remaining solvent was further removed at 90° C. and 9.8 kPa to obtain 183 g of the target compound (purity: 76%, compounds with molecular weights higher than that of the target compound: 24 area %).

The results of 1H-NMR analysis confirmed that a target compound (p-5) was obtained.

¹H-NMR analysis (400 MHZ, solvent: CDCl₃, reference material: tetramethylsilane)

0.30-0.40 (3H, m), 0.84 (1H, m), 0.90-1.00 (6H, m), 1.10 (1H, m), 1.76-2.02 (2H, m), 2.32 (1H, m), 2.58 (1H, m), 2.81-3.07 (4H, m), 3.57-4.05 (8H, m), 4.73-4.90 (4H, m), 6.50-7.12 (6H, m).

### <Example 9> (Synthesis of Compound p-6)

The reaction was performed in the same manner as in Example 4 except that a 1 L four-necked flask equipped with a thermometer, a stirrer, and a condenser, 97 g (0.31 mol) of 1,1′-bis(4-hydroxyphenyl)-3,3,5-trimethylcyclohexane, 48 g of 94% paraformaldehyde, 48 g of 2-aminoethanethiol, and 180 g of toluene were used, the temperature before the dropwise addition of amine was adjusted to 30° C., and stirring was further performed at 30° C., 40° C., and 50° C. each for 3 hours after completion of the dropwise addition of amine. The composition of the reaction solution was analyzed by GPC according to the above analysis method, revealing that the percentage of the target compound present in the reaction solution was 73 area %.

After completion of the reaction, alkali washing was performed using a 3% aqueous sodium hydroxide solution, and water washing was then performed until the pH of the reaction solution became 7 or less. Thereafter, toluene and water were removed by reduced-pressure distillation at 30° C. The pressure during the distillation was gradually reduced so as to finally reach 4.2 kPa. After the solvent was removed to some extent, the remaining solvent was further removed at 90° C. and 20 kPa. The composition containing the target compound was taken out, solidified by cooling, and then pulverized to obtain 188 g of the target compound (purity: 718, compounds with molecular weights higher than that of the target compound: 29 area %).

The results of 1H-NMR analysis confirmed that a target compound (p-6) was obtained.

¹H-NMR (400 MHZ, solvent: CDCl₃, reference material: tetramethylsilane)

0.25-0.44 (3H, m), 0.76-1.02 (7H, m), 1.11 (1H, dd), 1.36 (1H, d), 1.75-2.05 (2H, m), 2.33 (1H, brm), 2.59 (1H, brm), 2.77-3.22 (8H, m), 3.54-3.79 (4H, m), 3.86-4.07 (4H, m), 6.51-7.04 (6H, m), 9.07-10.3 (2H, brm).

1. A method for producing a benzoxazine compound represented by general formula (3), the method comprising performing a reaction of a bisphenol compound represented by general formula (1), a formaldehyde, and an amine compound represented by general formula (2) at 10° C. or higher and 80° C. or lower, 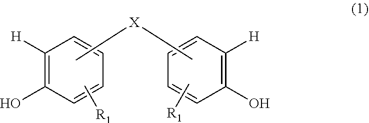
wherein R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b), 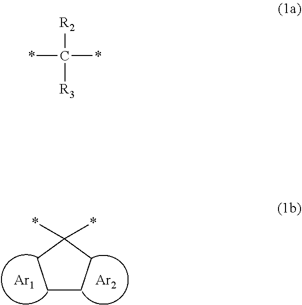
wherein, in general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R₃ are optionally bonded to each other to together form a cycloalkylidene group having 5 to 20 carbon atoms, Ar₁ and Ar₂ each independently represent an aryl group having 6 to 12 carbon atoms, and * represents a bonding position, 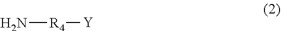
wherein R₄ represents a divalent group having 1 to 10 carbon atoms, and Y represents a hydroxy group or a thiol group, 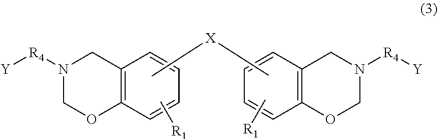
wherein R₁ and X are as defined in general formula (1), and R₄ and Y are as defined in general formula (2).
